(19) United States
(12) Patent Application Publication
(10) Pub. No.: US 2024/0285425 A1
(43) Pub. Date: Aug. 29, 2024

(54) COMPACT AND CANISTER-LESS URINE EXTRACTION DEVICE

(71) Applicants: Becky Donohoe, Berkeley, CA (US); Christopher Hudson, Fairfield, CA (US)

(72) Inventors: Becky Donohoe, Berkeley, CA (US); Christopher Hudson, Fairfield, CA (US)

(21) Appl. No.: 18/527,205

(22) Filed: Dec. 1, 2023

Related U.S. Application Data
(60) Provisional application No. 63/487,601, filed on Feb. 28, 2023.

(51) Int. Cl. A61F 5/44; A61M 1/00

(52) U.S. Cl. CPC A61F 5/4404; A61M 1/80

(57) ABSTRACT

The present disclosure provides a canister-less, compact and portable urine extraction and disposal device for extracting urine from individuals with limited mobility, disability, or a medical condition, which may be used as an extension to an external non-invasive urine capture device(s) to directly discharge urine into a urine disposal outlet, comprising an inlet tube, an outlet tube, a pump, a battery, a mounting bracket slot, and a charging port, wherein the urine extraction and disposal device extracts urine via the inlet tube and discards urine directly into a urine disposal outlet, such as a toilet, removing a need to carry a canister to collect urine to dispose later.

### CROSS-REFERENCE TO RELATED APPLICATIONS

This application claims benefit of and priority from U.S. Provisional Patent Application No. 63/487,601 filed on Feb. 28, 2023, entitled “A Compact and Canister-Less Urine Extraction Device,” the entire disclosure of which is hereby expressly incorporated by reference herein in its entirety.

### FIELD OF INVENTION

The invention generally relates to a urine extraction and disposal device. More specifically, the invention relates to a compact, portable, and canister-less urine extraction and disposal device and methods of using thereof.

### BACKGROUND

Individuals with limited mobility, disability, or immobility use a wheelchair or other accessibility vehicles to carry out their daily activities. Such individuals, especially females, need assistance for transferring to and from the toilet or commode chair for urination. The process can involve undressing, redressing, and/or transferring from their wheelchair onto and off a bed or dressing table, usually with the help of a caregiver or another person. There is a need for such individuals with limited mobility, disability, or immobility, such as females, to be able to remain seated in their wheelchair or accessibility vehicle to urinate with minimal effort and assistance. The existing female external catheter pump system(s) available in the market have a bulky vacuum pump that uses a canister to collect urine, making it impractical to use while such individuals are on the go in a wheelchair. The ability to urinate when and where an individual with limited mobility, disability, a medical condition or immobility wants without the help of a caregiver would significantly improve the life and independence of such individuals who struggle with many daily life activities. Thus, there is a need for a compact, canister-less, portable device facilitating direct urination while maintaining comfort and hygiene.

### SUMMARY

The following is a summary of the invention to provide a basic understanding of some aspects of the invention. This summary is not intended to identify key/critical elements of the invention or delineate the invention's scope. Its sole purpose is to present some concepts of the invention in a simplified form as a prelude to the more detailed description that is presented later.

The primary objective of the invention is to provide a portable, non-invasive and canister-less urine extraction and disposal device to assist an individual with limited mobility, disability, a medical condition or immobility to urinate directly in a urine disposal outlet and a method of its use thereof.

In various embodiments, the disclosed urine extraction and disposal device comprises an inlet tube, wherein one end of the inlet tube is attached to an extraction tube of an external non-invasive urine capture device, and another end of the inlet tube is attached to the disclosed urine extraction and disposal device through an inlet nozzle, wherein the device through the inlet tube extracts and withdraw urine from the extraction tube of the external urine capture device and discard the urine directly into a urine disposal outlet or similar outlet facilitating direct urination. The extraction tube is the tube of an external non-invasive urine collection or capture device, while the urine collection or capture device is properly positioned on a user of the urine extraction and disposal device, wherein the disclosed device functions as an extension to such external urine capture device.

In various embodiments, the external non-invasive urine capture device may be a female external catheter used to collect urine in a canister wherein the disclosed urine extraction and disposal device adds an extension and aid such external urine capture device(s) to remove the need to collect the urine in a canister but help the individuals using such external non-invasive urine capture devices(s) directly urinate in a urine disposal outlet.

In various embodiments, the inlet tube extracts or withdraws urine through the suction created via a pump present within or outside the disclosed urine extraction and disposal device. Once the pump is switched ON, the pump creates a suction to extract and withdraw urine from the extraction tube into the inlet tube.

In various embodiments, the disclosed device further comprises an outlet tube wherein one end of the outlet tube is placed directly into a urine disposal outlet, such as a toilet bowl or commode and the other end of the outlet tube is connected to the disclosed device via an outlet nozzle.

In various other embodiments, the disclosed device further comprises an inlet nozzle to attach the inlet tube to the device, an outlet nozzle to attach an outlet tube to the device, a hose bib, a charging port, a switch to operate the device, a pump, a battery, and a mounting bracket slot. The inlet nozzle and the outlet nozzle help maintain the flow of the fluid, such as urine, within the inlet tube and the outlet tube.

In various embodiments, the urine extraction and disposal device help a person with limited mobility a medical condition or immobility to directly urinate into a urine disposal outlet, such as a toilet or a restroom facility.

In various embodiments, the disclosed device can be mounted to a wheelchair or another accessibility vehicle, wherein an individual on the wheelchair does not have to carry the device separately, but the device can be mounted to the wheelchair or the accessibility device without affecting any functionality, stability, or operation of the wheelchair or the accessibility vehicle. The disclosed device may also be mounted to a hospital bed, user's bed, a wall or other such flat surfaces, giving access to a hygienic, non-invasive urine extraction and disposal facility to an individual with limited mobility, disability, or immobility.

In various embodiments, the disclosed device comprises a battery to power the device during its use. The battery may be a chargeable battery, a non-chargeable battery, a reusable battery, or a solar-powered battery.

In many embodiments, the disclosed device can be used by an individual using a wheelchair by attaching or inserting the inlet tube of the disclosed device to or within the extraction tube of an external non-invasive urine capture device(s) used by such individual and placing one end of the outlet tube in an output reservoir or a urine disposal outlet, wherein the outlet may be a toilet bowl, a bottle, or a bag such that the outlet tube is placed directly into one of the outlet reservoirs of choice.

In various embodiments, the device lets an individual pump urine up to a distance of 30 feet, preferably 20 feet. This benefits the user in that if they are in a public or private place where a toilet is not easily accessed because of some type of barrier, they can be at an extended distance and still be able to urinate directly into a toilet. In various other embodiments, the disclosed device can also be used by an individual who is in bed for extended periods and a bathroom toilet is within the distance of the outlet tube length from where the person is in bed, the user can urinate directly into the toilet and not be restricted to a canister or container with limited capacity. This eliminates the need to continually empty and clean a canister or container during periods of time while bedridden.

In many embodiments, the length of the inlet tube is shorter than the length of the outlet tube. The inlet and outlet tubes may be made of plastic or other flexible, liquid-proof material.

In the description, for purposes of explanation and not limitation, specific details are set forth, such as particular embodiments, procedures, techniques, etc., in order to provide a thorough understanding of the present technology. However, it will be apparent to one skilled in the art that the present technology may be practiced in other embodiments that depart from these specific details.

While specific embodiments of, and examples for, the process described above for illustrative purposes, various equivalent modifications are possible within the scope of the system, as those skilled in the relevant art will recognize.

For example, while steps are presented in a given order, alternative embodiments may perform routines having steps in a different order, and some processes or steps may be deleted, moved, added, subdivided, combined, and/or modified to provide alternative or sub-combinations. Each of these steps may be implemented in a variety of different ways. Also, while steps are sometimes shown as being performed in series, these steps may be performed in parallel or at different times.

While various embodiments have been described above, it should be understood that they have been presented by way of example only and not limitation. The descriptions are not intended to limit the scope of the present technology to the particular forms set forth herein. On the contrary, the present descriptions are intended to cover such alternatives, modifications, and equivalents as may be included within the spirit and scope of the present technology as appreciated by one of ordinary skill in the art. Thus, the breadth and scope of a preferred embodiment should not be limited by any of the above-described exemplary embodiments.

### BRIEF DESCRIPTION OF THE DRAWINGS

The accompanying drawings, where like reference numerals refer to various embodiments or steps of the methods, together with the detailed description below, are incorporated in and form part of the specification, and serve to further illustrate embodiments of concepts that include the claimed disclosure, and explain various principles and advantages of those embodiments.

The device and methods disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1(a) illustrates a back view of the disclosed urine extraction and disposal device according to the present disclosure.

FIG. 1(b) illustrates a front view of the disclosed urine extraction and disposal device according to the present disclosure.

FIG. 2 illustrates an exploded view of the disclosed urine extraction and disposal device according to the present disclosure.

FIG. 3 illustrates an exemplary use of the disclosed urine extraction and disposal device to directly dispose of urine in a variety of outlets according to the present disclosure.

FIG. 4(a) illustrates an exemplary embodiment of using a small bag or backpack hung on a wheelchair according to the present disclosure.

FIG. 4(b) illustrates an exemplary embodiment of the disclosed urine extraction and disposal device mounted to a wheelchair, according to the present disclosure.

FIG. 4(c) illustrates an exemplary embodiment of using the disclosed urine extraction and disposal device while an individual is lying on a bed, according to the present disclosure.

FIG. 4(d) illustrates a wall-mounted position of the disclosed urine extraction and disposal device according to the present disclosure.

The exemplary embodiments described and illustrated herein should be applicable to all urine extraction and disposal devices.

### DETAILED DESCRIPTION

While the presently disclosed device and methods are susceptible of embodiment in many different forms, there are shown in the drawings and will herein be described in detail several specific embodiments with the understanding that the present disclosure is to be considered as an exemplification of the principles of the present technology and is not intended to limit the technology to the embodiments illustrated.

In summary, the present disclosure provides a urine extraction and disposal device that may work as an extension to an external non-invasive urine capture device(s) for extracting urine and then discarding it directly into a urine disposal outlet. In many embodiments, the present disclosure provides a canister-less, compact and portable urine extraction and disposal device for extracting or withdrawing urine from individuals with limited mobility, disability, or a medical condition who utilize external non-invasive urine capture device(s) and dispose or discard directly into a urine disposal outlet, such as a toilet. The urine extraction and disposal device comprises an inlet tube, an inlet nozzle, an outlet tube, an outlet nozzle, a pump, a battery, a motor, a mounting bracket slot, a hose bib, and a charging port, wherein the urine extraction and disposal device extracts urine using the inlet tube and discards urine through the outlet tube directly into a urine disposal outlet, such as a toilet and removes the need for carrying a canister or other container or a bag to collect urine for later disposal. Thus, the urine extraction and disposal device, according to the present invention, obviates the need for a separate device or a container to collect to dispose later, but instead, a user can use a regular toilet and dispose of urine directly into the regular toilet or any other outlet to dispose of urine.

In various embodiments, the disclosed urine extraction and disposal device comprises an enclosure, which may be a hollow enclosure, surrounded by a rigid but lightweight frame defining an interior cavity therein. The enclosure comprises a pair of a first flat surface and a pair of a second flat surface, forming a hollow frame defining the internal cavity therein. The urine extraction and disposal device is a compact structure, making it easy to use and transport. The frame may be formed in a compact geometrical shape, such as rectangular with two parallel sides or a square.

In various embodiments, the inlet tube can be inserted into an extraction tube of an external non-invasive urine capture device(s) when the external non-invasive urine capture device(s) is properly positioned on a user with a disability, limited ability, or a medical condition, such that the inlet tube extracts urine through the extraction tube using the suction created by a pump when the urine extraction and disposal device is switched ON to discard directly into the urine disposal outlet or facility.

In various embodiments, the outlet tube is placed directly into a urine disposal outlet for urine disposal, wherein one end of the outlet tube disposes or discards the urine collected through the inlet tube directly into a urine disposal outlet. The urine disposal outlet may be a toilet bowl, a bottle, a bag, or a container to hold urine. The urine capturing and disposal device may use suction or pressure to pump urine from the outlet tube into the urine disposal outlet.

In various embodiments, the urine extraction and disposal device further comprises a mounting bracket slot wherein the mounting bracket slot provides a slot for mounting the device to any flat and stable surface. The disclosed urine extraction and disposal device may be mounted on an armrest or an accessory bracket of a wheelchair so that an individual using a wheelchair can urinate directly into a toilet while sitting and without moving to an uncomfortable position or leaving the wheelchair for urination. The disclosed urine extraction and disposal device may be mounted on other hard surfaces or flat surfaces having a mounting bracket or accessories such as a hook to hold the urine extraction and disposal device, making it convenient to be used at various places by the user with limited mobility, disability, or immobility. The hard surface or flat surface may be a wall, a bed, a side of the wheelchair, a door or other such flat surfaces. The mounting bracket may be made of the same material as the urine extraction and disposal device or other sturdy material to hold the weight of the urine extraction and disposal device while in a mounted position. The mounting bracket may be a support hardware used to hold, attach, mount, or secure the disclosed urine extraction and disposal device for use.

In various embodiments, the urine extraction and disposal device may be battery operated, wherein the battery may be a rechargeable battery. The use of a rechargeable battery makes the urine extraction and disposal device portable such that an individual user may recharge the battery and can transport it to places such as mountains, outdoor vacations, concerts, or while traveling, and urinate without worrying about power or durability. In various other embodiments, the battery may be a single-use battery or a solar-powered battery.

In various embodiments, the disclosed urine extraction and disposal device comprises a pump, wherein the pump creates a suction necessary to extract and withdraw urine through the inlet tube or discard urine from the outlet tube directly into a urine disposal outlet. The pump may be powered by a pump motor located within the internal cavity of the urine extraction and disposal device. The pump motor powers the pump when the urine extraction and disposal device is switched ON.

In various embodiments, the present disclosure provides an exemplary method of using the urine extraction and disposal device, comprising mounting the urine extraction and disposal device on a flat surface, inserting or placing an open end of an inlet tube of the urine extraction and disposal device into an extraction tube of an external non-invasive urine capture device(s); placing an open end of an outlet tube into a urine disposal outlet; withdrawing urine from the external non-invasive urine capture device(s) into the inlet tube through a suction created by a pump present within the urine extraction and disposal device; transporting the urine from the inlet tube into an outlet tube to dispose urine into a urine disposal outlet through the open end of the outlet tube; and removing the outlet tube from the urine disposal outlet once the urination is complete. In various other embodiments, the present disclosure provides an exemplary method of using the urine extraction and disposal device by an individual using a wheelchair, comprising mounting the urine extraction and disposal device on a flat surface, such as an armrest or an accessory bracket of a wheelchair; inserting an inlet tube of the urine extraction and disposal device into an extraction tube of an external non-invasive urine capture device(s), wherein the external non-invasive urine capture device(s) is positioned properly on the individual; placing an outlet tube into a urine disposal outlet; switching on the urine extraction and disposal device to start a pump, wherein the pump creates suction necessary to withdraw urine from an external non-invasive urine capture device(s) into the inlet tube; disposing urine through the outlet tube directly into the urine disposal outlet; and removing the outlet tube from the urine disposal outlet once the urination is complete.

In various embodiments, the urine extraction and disposal device, in accordance with the present invention, may be used to collect urine from humans, especially from humans with limited mobility, medical conditions, disability or bedridden.

In many embodiments, the disclosed urine extraction and disposal device, in accordance with the present invention, is a compact portable device that can be hand-held, surface-mounted, or toted in a small bag or backpack.

In various embodiments, the urine extraction and disposal device, according to the present invention, provides independence for individuals with limited mobility or disability wherein the individual can carry the urine extraction and disposal device and use the urine extraction and disposal device for urination almost anywhere, wherein the urine extraction and disposal device allows such individuals to discretely urinate while seated in a wheelchair. The urine disposal outlet may be a toilet, a toilet bowl, a commode, a bag, a bottle or such reservoirs.

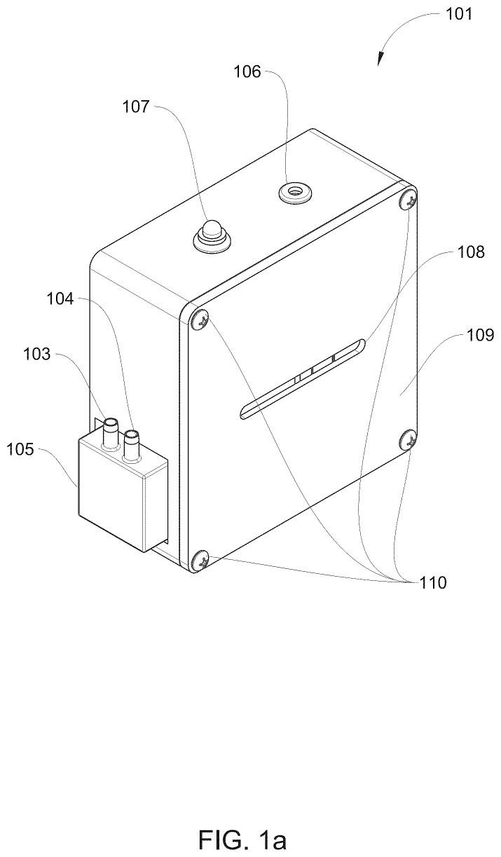

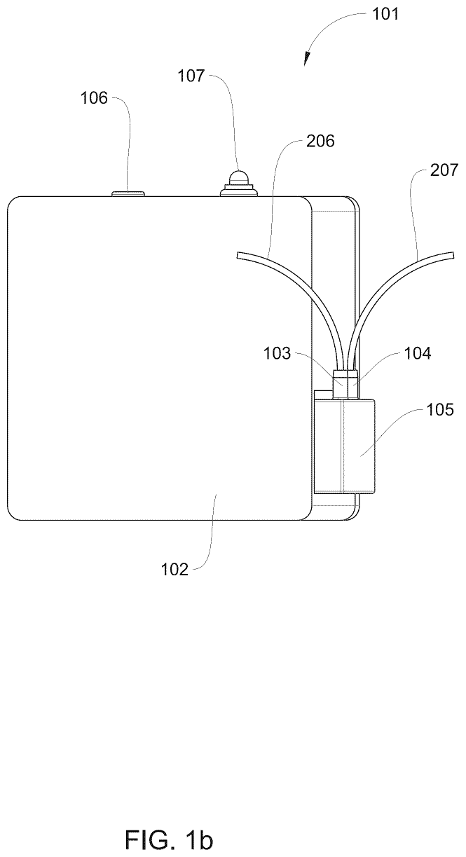

FIG. 1(a) illustrates a back view of the disclosed urine extraction and disposal device (101) according to the present disclosure. As shown in FIG. 1(a), the urine extraction and disposal device comprises an inlet nozzle (103) and an outlet nozzle (104). As shown in FIG. 1, the urine extraction and disposal device (101) comprises a pair of a first flat surface, such as a back cover plate (109) and a pair of a second flat surface (102), wherein the first flat surface and the second flat surface join to form a frame structure defining an internal cavity of the urine extraction and disposal device (101). The first flat surface (109) may be larger in size than the second flat surface (102), or the first flat surface (109) and the second flat surface (102) may be of the same size, wherein the pair of the first flat surface (109) and the pair of the second flat surface (102) may join to form a geometrical shape forming the exterior walls of the disclosed urine extraction and disposal device (101). The back cover plate (109) is held in place by back plate screws (110) holding the back plate in position. FIG. 1 further illustrates that the urine extraction and disposal device (101) comprise a charger port (106) wherein the charger port (106) is used to charge batteries, and a battery (203) located within the internal cavity of the urine extraction and disposal device (101). The urine extraction and disposal device (101) further comprises an ON/OFF button (107) to operate the disclosed urine extraction and disposal device. FIG. 1 further shows that the device comprises a mounting bracket slot (108) used to mount the disclosed device to a mounting bracket on a flat and stable surface. The mounting bracket slot (108) holds the disclosed device in a stable position while the disclosed urine extraction and disposal device is in use. The mounting bracket may be a wall mount bracket, a bed mount bracket or a wheelchair accessory bracket. The urine extraction and disposal device further comprises a hose bib (105) holding the inlet nozzle (103) and the outlet nozzle (104).

FIG. 1(b) illustrates a front view of the disclosed urine extraction and disposal device according to the present disclosure. As shown in FIG. 1(b), the outlet tube (207) is affixed, attached, or inserted into the outlet nozzle (104), and an inlet tube (206) is affixed, attached or inserted into the inlet nozzle (103). FIG. 1(b) further shows a front cover plate (102) or the front side of the disclosed urine extraction and disposal device. The inlet tube (206) and the outlet tube (207) may direct the flow of fluid, such as urine, in and out of the tubes, wherein the rate or speed of the flow of urine through the inlet or outlet tubes is controlled by the inlet nozzle (103) and the outlet nozzle (104), respectively.

In various embodiments, the pair of the first flat surface, such as a back cover plate (109), and the second flat surface (102), may join to form the urine extraction and disposal device as a rectangle or a square-shaped device. The urine extraction and disposal device may have a dimension of 5¼″ L 5¼″ W×2½″ with a 1¾″ square opening on one or either side of the device for pump, connection port, battery, pump, and other housing protrusion. The urine extraction and disposal device may be made of plastic, wood, acrylic, or other medical-grade material.

The inlet tube may have a diameter ranging from ⅛″-1″ such that the inlet tube (206) can be inserted into an external non-invasive urine capture device(s) used by a user, such as a non-invasive female external catheter or urine drainage bag. The outlet tube (201) may have a diameter ranging from 3/16″-1″ wherein the outlet tube (207) is directly in contact with a urine disposal outlet, wherein the outlet tube (207) releases the urine directly into the urine disposal outlet. The inlet and the outlet tubes may vary in length.

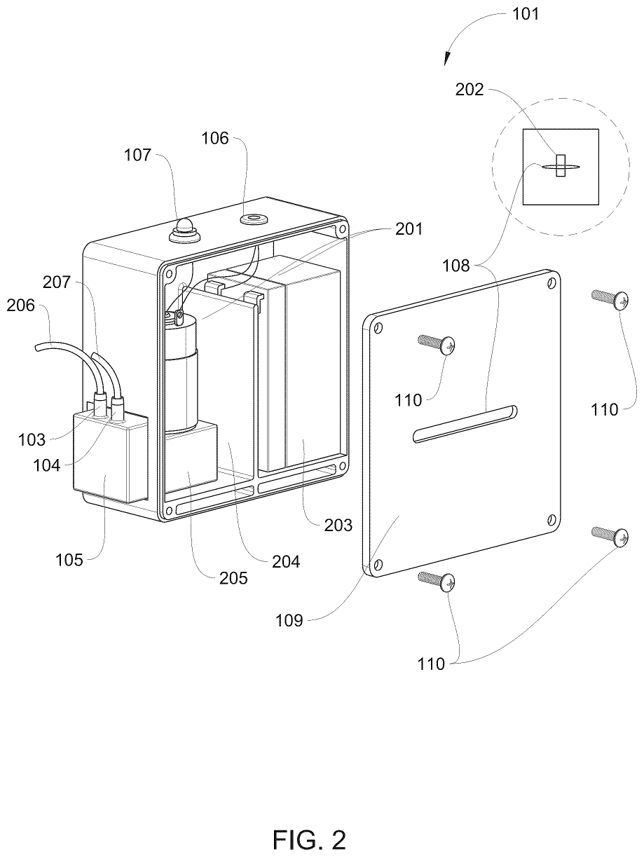

FIG. 2 illustrates an exploded view of the disclosed urine extraction and disposal device according to the present disclosure. As shown in FIG. 2, the back cover plate is separated from the rest of the urine extraction and disposal device by loosening the back cover plate screws (110) showing inside or internal cavity of the disclosed urine extraction and disposal device. The device comprises a wiring harness (201), an example universal mounting bracket (202), a battery (203), and an interior component divider plate (204), dividing the inner cavity of the disclosed urine extraction and disposal device to place the battery in a section separate from the other section within the inner cavity. The urine extraction and disposal device further comprises a motor (205) with a wiring harness (201) necessary to operate the disclosed urine extraction and disposal device.

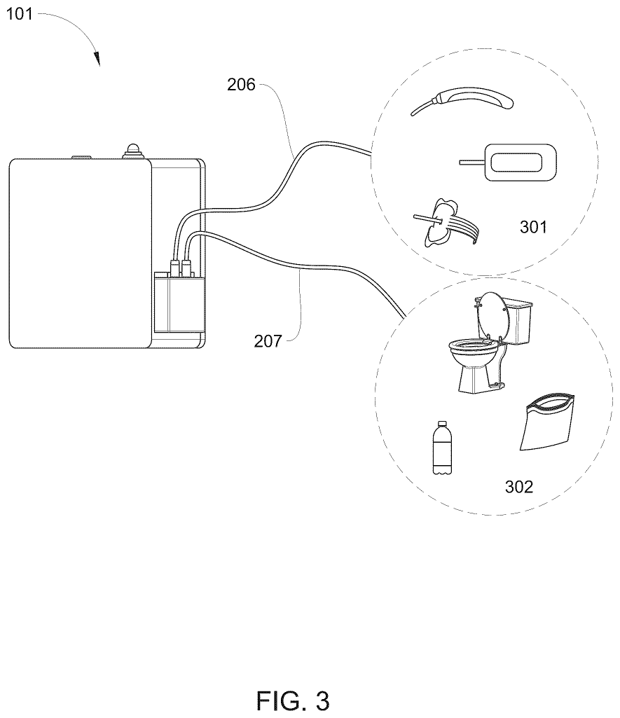

FIG. 3 illustrates an exemplary use of the disclosed urine extraction and disposal device to directly discard of urine into a variety of urine disposal outlets according to the present disclosure. FIG. 3 shows a few examples of the inlet and the outlet options or forms that can be used together with the disclosed device for urine extraction and disposal, which is convenient, non-invasive and does not need an individual to carry a canister or move from its current position or move to urinate. 301 provides a few examples of the external non-invasive urine capture devices, such as a female external catheter, and 302 shows a few examples of urine disposal outlet, such as a bottle, a bag, or a toilet.

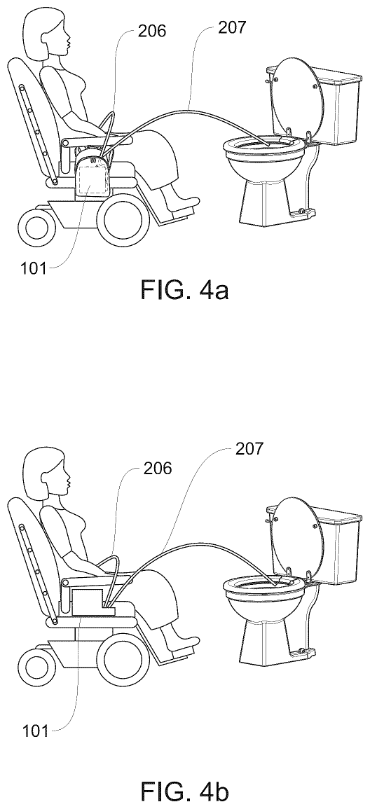

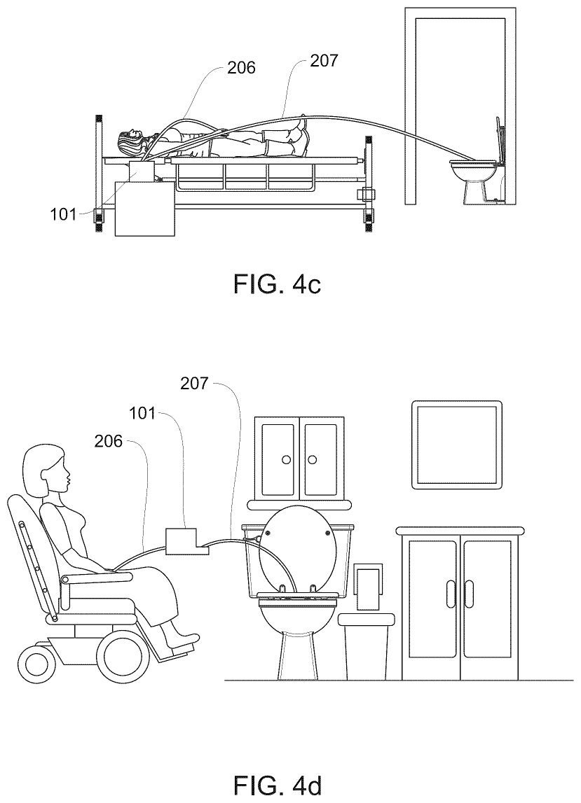

FIG. 4(a) illustrates an exemplary embodiment of using a small bag or backpack hung on a wheelchair according to the present disclosure. As shown, the disclosed urine extraction and disposal device (101) can be put in a bag holding the urine extraction and disposal device so an individual on the wheelchair can attach the bag with the urine extraction and disposal device on one side or an arm of the wheelchair. The inlet tube (206) and the outlet tube (207) will be hanging out of the bag only during use. This way, the individual using the bag can keep the tubes hygienic and less cumbersome to manage. During use, the user can put an inlet tube (206) to insert the inlet tube into an external non-invasive urine capture device wherein the inlet tube, through the suction created by a motor and a pump, withdraws urine from the extraction tube of the external urine capture device, such as a female external catheter, into the inlet tube and then discard it directly into a urine disposal outlet, such as a toilet through the outlet tube (207). The length of the inlet tube (206) is usually shorter than the length of the outlet tube (207). This way, the user can directly urinate into a toilet by directly putting an open end of the outlet tube (207) near the open mouth of the toilet so that urine extracted from an individual through the inlet tube ends in a toilet bowl or other urine disposal outlets, obviating a need to hold the urine in a container or a bag.

FIG. 4(b) illustrates an exemplary embodiment of the disclosed urine extraction and disposal device (101) mounted to a wheelchair. As shown in FIG. 4(b), the disclosed device is directly mounted to a wheelchair used by a user. The mounting bracket of the device through the slot can be at a position which does not affect the function, comfort, or operations of the wheelchair or cause any inconvenience to the user of the wheelchair.

FIG. 4(c) illustrates an exemplary embodiment of using the disclosed device (101) while an individual is lying on the bed, according to the present disclosure. As shown in FIG. 4(c), the disclosed device (101) can be used in a hospital setting wherein an individual, such as a patient, is lying on the hospital bed wherein the inlet tube (206) is inserted into an extraction tube of an external urine capture device used by the patient to extract and withdraw urine and then directly dispose the urine into a toilet through the outlet tube (207), which obviates the need to hold the urine in a bag for late disposal. The urine extraction and disposal device (101) can be used in various similar settings such as home, nursing home, elder facility, medical facility, or other facilities assisting individuals who are immobile, bedridden or in bed for extended periods to urinate directly into a urinal, such as a toilet.

FIG. 4(d) illustrates a wall-mounted position of the disclosed urine extraction and disposal device (101) according to the present disclosure. As shown in FIG. 4(d), the disclosed urine extraction and disposal device (101) can be mounted on a wall, such as a bathroom wall, for an individual's use. The inlet tube (206) from the disclosed urine extraction and disposal device is inserted into an extraction tube of an external urine capture device used by the individual, switch the urine extraction and disposal device ON so the urine extraction and disposal device can suction urine from the extraction tube into an inlet tube and dispose of the urine directly into the toilet through the outlet tube (207) without the individual moving from their chair and sitting on the toilet seat for urination. The disclosed urine extraction and disposal device can also be mounted in places providing handicapped bathroom facilities or accessibility facilities.

In the description, for purposes of explanation and not limitation, specific details are set forth, such as particular embodiments, procedures, techniques, etc., in order to provide a thorough understanding of the present technology. However, it will be apparent to one skilled in the art that the present technology may be practiced in other embodiments that depart from these specific details.

While specific embodiments of, and examples for, the device and methods are described above for illustrative purposes, various equivalent modifications are possible within the scope of the system, as those skilled in the relevant art will recognize. For example, while steps are presented in a given order, alternative embodiments may perform routines having steps in a different order, and some steps may be deleted, moved, added, subdivided, combined, and/or modified to provide alternative or sub-combinations. Each of these steps may be implemented in a variety of different ways. Also, while steps are at times shown as being performed in series, these steps may instead be performed in parallel or may be performed at different times.

While various embodiments have been described above, it should be understood that they have been presented by way of example only and not limitation. The descriptions are not intended to limit the scope of the present technology to the particular forms set forth herein. To the contrary, the present descriptions are intended to cover such alternatives, modifications, and equivalents as may be included within the spirit and scope of the present technology as appreciated by an ordinary skill in the art. Thus, the breadth and scope of a preferred embodiment should not be limited by any of the above-described exemplary embodiments.

What is claimed is:

1. A urine extraction and disposal device comprising:
an inlet tube;
an outlet tube;
a pump;
a battery;
a mounting bracket slot; and
a charging port, wherein the urine extraction and disposal device extracts urine through an extraction tube on an external urine capture device and disposes the urine directly into a urine disposal outlet through the outlet tube.

2. The urine extraction and disposal device of claim 1, wherein one end of the inlet tube is inserted into an extraction tube of an external urine capture device, and another end of the inlet tube is attached to the urine extraction and disposal device through an inlet nozzle.

3. The urine extraction and disposal device of claim 2, wherein the urine extraction and disposal device facilitate direct urination for individuals with limited mobility by extracting urine from the extraction tube of the external urine capture device into the inlet tube and discarding the urine directly into a urine disposal outlet through the outlet tube.

4. The urine extraction and disposal device of claim 3, the inlet tube extracts urine through the suction created via the pump present within the urine extraction and disposal device.

5. The urine extraction and disposal device of claim 1, wherein the external urine capture device is a female external catheter.

6. The urine extraction and disposal device of claim 1, wherein an open end of the outlet tube is placed directly into the urine disposal outlet.

7. The urine extraction and disposal device of claim 1, wherein the device is mounted to a wheelchair.

8. The urine extraction and disposal device of claim 1, wherein the device is mounted to a wall.

9. A method of using a urine extraction and disposal device comprising:
mounting the urine extraction and disposal device to a flat surface;
inserting an inlet tube of the urine extraction and disposal device into an extraction tube of an external urine capture device;
placing an outlet tube into a urine disposal outlet;
withdrawing urine from the external urine capture device into the inlet tube through a suction created by a pump present within the urine extraction and disposal device;
discarding the urine through the outlet tube into a urine disposal outlet; and
removing the inlet tube from the extraction tube of the external urine capture device and the outlet tube from the urine disposal outlet once the urination is complete.

10. The method of claim 9, wherein one end of the inlet tube is attached to an extraction tube of an external urine capture device and another end of the inlet tube in attached to the urine extraction and disposal device through an inlet nozzle.

11. The method of claim 9, wherein the urine extraction and disposal device facilitate direct urination for individuals with limited mobility by extracting urine from the extraction tube of the external urine capture device into the inlet tube and discarding the urine directly into a urine disposal outlet through the outlet tube.

12. The method of claim 9, wherein the external urine capture device is a female external catheter.

13. The method of claim 9, wherein the urine extraction and disposal device is mounted to a wheelchair.

14. The method of claim 9, wherein an open end of the outlet tube is placed directly into a urine disposal outlet.

15. A method of using the urine extraction and disposal device by a wheelchair-bound individual comprising:
mounting the urine extraction and disposal device to an armrest of the wheelchair;
inserting an inlet tube of the urine extraction and disposal device into an extraction tube of an external urine capture device,
placing an outlet tube into a urine disposal outlet;
withdrawing urine from the external urine capture device into the inlet tube through a suction created by a pump present within the urine extraction and disposal device;
collecting urine through the outlet tube into a urine disposal outlet; and
removing the inlet tube from the extraction tube of the external urine capture device and the outlet tube from the urine disposal outlet once the urination is complete.

16. The method of claim 15, wherein one end of the inlet tube is attached to an extraction tube of an external urine capture device and another end of the inlet tube is attached to the urine extraction and disposal device through an inlet nozzle.

17. The method of claim 15, wherein the urine extraction and disposal device facilitate direct urination for individuals with limited mobility by extracting urine from the extraction tube of the external urine capture device into the inlet tube and discarding the urine directly into a urine disposal outlet through the outlet tube.

18. The method of claim 15, wherein the external urine capture device is a female external catheter.

19. The method of claim 15, wherein the urine extraction and disposal device is mounted to a wheelchair.

20. The method of claim 15, wherein an open end of the outlet tube is placed directly into the urine disposal outlet.
